(19) United States
(12) Patent Application Publication
(10) Pub. No.: US 2023/0076197 A1
(43) Pub. Date: Mar. 9, 2023

(54) APPLICATION OF HYPERICUM JAPONICUM THUMB EXTRACT IN THE PREPARATION OF A DRGU AGAINST THE BLUE-EAR DISEASE

(71) Applicant: SUN YAT-SEN UNIVERSITY, Guangzhou (CN)

(72) Inventors: Weiwei SU, Guangzhou (CN); Yonggang WANG, Guangzhou (CN); Peibo LI, Guangzhou (CN); Hongyu RAO, Guangzhou (CN); Hao WU, Guangzhou (CN)

(73) Assignee: SUN YAT-SEN UNIVERSITY, Guangzhou (CN)

(21) Appl. No.: 17/962,560

(22) Filed: Oct. 10, 2022

(30) Foreign Application Priority Data
Aug. 10, 2021 (CN) 202110915261.6

(51) Int. Cl. A61K 36/185; A61P 31/14

(52) U.S. Cl. CPC A61K 36/185; A61P 31/14; A61K 2236/333

(57) ABSTRACT

An application of HJT aqueous extract in the preparation of drugs against a Blue-ear disease is provided. For the first time, it is found in the present invention that HJT extract has a significant effect of preventing and treating the Blue-ear disease. Specifically, through experiments of qRT-PCR and immunofluorescence techniques, the present invention has proved that HJT extract can significantly inhibit infection and replication of PPRSV with significant antiviral effect, so that it is expected to become a new type of biologically active substance for prevention and treatment of Blue-ear disease. The product prepared by the method can be used as natural antiviral drugs for the prevention and treatment of the Blue-ear disease, realizes the comprehensive utilization of HJT plants, and improves its value, which has a good application prospect.

### CROSS REFERENCE TO THE RELATED APPLICATIONS

This application is based upon and claims priority to Chinese Patent Application No. 202110915261.6, filed on Aug. 10, 2021, the entire contents of which are incorporated herein by reference.

### TECHNICAL FIELD

The present disclosure belongs to the field of traditional Chinese medicine pharmacy, in particular to an application of Hypericum japonicum thumb (HUT) extract in the preparation of a drug against the Blue-ear disease.

### BACKGROUND

Porcine reproductive and respiratory syndrome (PRRS), also known as the Blue-ear disease, is caused by PRRS virus (PRRSV). PRRS mainly causes abortion, stillbirth, mummified fetus, weak litter in pregnant sows, and respiratory symptoms of pigs of all ages, especially piglets. The characteristic of PPRS was interstitial pneumonia with extremely high mortality, which is a highly contactable and globally important infectious disease.

The prevention and control of PRRSV is currently a difficult problem all over the world. The difficulties in prevention and control of PRRSV mainly lie in the following aspects: (1) the mutation is rapid, and the vaccine has almost no cross-protection; (2) the body's immune system is destroyed, and the immune response is suppressed; (3) the body is stimulated to produce low-titer antibodies, which is conducive to the further proliferation of the virus; (4) the persistent infection of the virus can last for up to 5 months, which is easy to cause mixed infection.

At present, there are mainly inactivated vaccines and attenuated vaccines for the prevention and control of PRRSV, wherein the inactivated vaccines have the following disadvantages: (1) large doses of inoculation or an application of concentrated antigens are required, and the immunization period is short, and intensive vaccination is often required; (2) inactivated vaccines can only induce systemic humoral responses, but it is difficult to induce cellular immunity; (3) it takes 2 to 3 weeks to produce complete immunity, which is not beneficial to emergency vaccination and to reduction of vaccine costs; (4) there is the possibility of incomplete inactivation and virus dispersal. Therefore, attenuated vaccines are commonly used in clinical practice. However, attenuated vaccines have the risk of virulence reversion, recombination and potential infection. As vaccines expose more and more problems in the prevention and control of PRRSV, it is urgent to find and develop an antiviral drug with anti-PPRSV.

HJT is the whole plant of the Hypericum japonicum of the genus Hypericum in the Garcinaceae family, which is widely distributed in the Yangtze River valley and all parts south of China. It has the functions of clearing away heat and detoxification, promoting blood circulation and reducing swelling, and is mainly used for the treatment of anti-oxidation, anti-liver damage and anti-virus.

### SUMMARY

The technical problem to be solved by the present invention is to overcome the defects and deficiencies of the existing prevention and treatment technology of PRRS, and to provide the application of HJT extract in the preparation of drugs against Blue-ear disease.

It is found that HJT has significant antiviral effect upon PRRSV, and shows a good application prospect for the prevention and treatment of PRRSV in the present invention.

The purpose of the present invention is to develop the new application of HJT in the field of pharmacy.

Therefore, the first object of the present invention is to provide an application of HJT aqueous extract, HJT ethanol extract or HJT ethanol aqueous solution extract in the preparation of a drug against PRRSV.

Preferably, it is an application in the preparation of drugs against Blue-ear disease.

Preferably, it is the HJT aqueous extract, which can be used to develop a more active extract for preparation of anti-PRRSV drugs.

Preferably, the HJT aqueous extract, the HJT ethanol extract or the HJT ethanol aqueous solution extract are HJT herb extracted by water, ethanol or ethanol aqueous solution respectively, and the HJT aqueous extract, the HJT ethanol extract or the HJT ethanol aqueous solution extract are obtained by recovering the solvent.

Further preferably, the ethanol aqueous solution is an ethanol aqueous solution with a volume fraction of 30%, 60% or 95%.

Preferably, the HJT aqueous extract or the HJT ethanol aqueous solution extract is obtained by adding the water decoction of HJT to the macroporous resin column, eluting sequentially with water and ethanol aqueous successively, and then recovering the solvent.

The sequentially eluting the macroporous adsorption resin column with the water and the ethanol aqueous solution is water, and an ethanol aqueous solution with a volume fraction of 20% and 60%.

In the present invention, the extract of HJT was separated, and each extract for the prevention and treatment of PRRS was screened. The experimental results show that: HJT aqueous extract, ethanol extract with a volume fraction of 30%, ethanol extract with a volume fraction of 60% and ethanol extract with a volume fraction of 95% all have strong effects on the prevention and treatment of PRRS, wherein HJT aqueous extract had the best effect.

Further, in the present invention, the HJT aqueous extract was splitting, and the anti-PPRSV pharmacological action of extracts with different polarities were screened. The experimental results showed that the extracts eluted by water, and ethanol with a volume fraction of 20% and 60% all had strong anti-PPRSV effects, among which the aqueous extracts eluted by ethanol with a volume fraction of 60% from HJT aqueous extract had the best effect.

In addition, the second object of the present invention is to provide a drug against PPRSV and/or a drug for prevention and treatment of Blue-ear disease, which contains HJT aqueous extract or HJT ethanolic extract or HJT aqueous ethanolic extract as an active ingredient.

The dosage form of the medicine can be prepared into different required dosage forms, such as powder, oral liquid or injection, etc.

The present invention has the following beneficial effects:

For the first time, it is found in the present invention that HJT extract has a significant effect of preventing and treating the Blue-ear disease. Specifically, through qRT-PCR and immunofluorescence techniques, the present invention has proved that HJT extract can significantly inhibit infection and replication of PPRSV, so that it is expected to become a new type of biologically active substance for prevention and treatment of Blue-ear disease. The products prepared by this method can be used as natural antiviral drugs for the prevention and treatment of the Blue-ear disease, and realizes the comprehensive utilization of HJT plants and increase its added value. Thus, this invention has a good application prospect.

### BRIEF DESCRIPTION OF THE DRAWINGS

FIG. 1 shows the cytotoxicity of HJT extracts with different polarities were detected by AlamarBlue on Marc-145 cells, in which S-06-0 represents water eluent, S-06-20% represents ethanol eluent with a volume fraction of 20%, S-06-60% represents ethanol eluent with a volume fraction of 60%.

FIG. 2 shows that Marc-145 cells infected with PRRSV at different multiplicity of infection (MOIs) were treated with HJT extracts of different polarities for 24 hours, and the number of PRRSV infection was detected by immunofluorescence, wherein PBS represents PBS control, S-06-0 represents water eluate, S-06-20% represents ethanol eluent with a volume fraction of 20%, and S-06-60% represents ethanol eluent with a volume fraction of 60%.

FIG. 3 shows that the Marc-145 cells were infected with PRRSV-EGFP strain (MOI=0.1), and different concentrations of HJT extracts with different polarities were added, and then cultured in an incubator under the condition of 5% CO₂, 37° C. for 24 hours, and the number of PRRSV infections were detected by immunofluorescence, wherein PBS represents PBS control, S-06-0 represents water eluate, S-06-20% represents ethanol eluent with a volume fraction of 20%, and S-06-60% represents ethanol eluent with a volume fraction of 60%.

FIG. 4 shows that the Marc-145 cells were infected with PRRSV (MOI=0.1), and different concentrations of HJT extracts with different polarities were added, and then cultured in an incubator under the condition of 5% CO₂, 37° C. for 24 hours, and the N protein mRNA level of PRRSV was detected by qRT-PCR, wherein S-06-20% represented ethanol eluent with a volume fraction of 20%, and S-06-60% represented ethanol eluent with a volume fraction of 60%.

### DETAILED DESCRIPTION OF THE EMBODIMENTS

The present invention is further described below with reference to the figures of the specification and specific embodiments, but the embodiments do not limit the present invention in any form. Unless otherwise specified, the reagents, methods and equipment used in the present invention are conventional reagents, methods and equipment in the art.

Unless otherwise specified, the reagents and materials used in the following examples are commercially available.

Statistical analysis of the following embodiments of the present invention are as follows: all cell experiments were repeated for at least 3 times independently, and the results were expressed by means and standard errors using one-way ANOVA and T-test analysis. All statistical analyses used P<0.05 as the test standard with significant statistical difference, and the analysis software was SPSS 16.0 and GraphPad Prism 7.

### Embodiment 1: Preparation of HJT Aqueous Extract and its Experimental Samples

HJT was purchased from Wuzhou City, Guangxi Province. The dry whole plant of HJT was taken, washed to remove the weeds and soil of the grass, and sliced into pieces of about 1-2 cm. About 500 g of the dry whole plant was extracted with 12 times the amount of boiled water for 0.5 h each time to extract three times. The extracting solution was combined, filtered, and concentrated to obtain an extract with a relative density of 1.18-1.20. An appropriate amount of dextrin was added (adding the extract and dextrin at a mass ratio of 1:1), and dried in an oven at 120° C., and then pulverized to obtain the extract.

### Embodiment 2: Preparation of HJT 30% Ethanol Extract and its Experimental Samples

HJT was purchased from Wuzhou City, Guangxi Province. The dry whole plant of HJT was taken, washed to remove the weeds and soil of the grass, and sliced into pieces of about 1-2 cm. About 500 g of the dry whole plant was extracted with 12 times the amount of ethanol aqueous solution with a volume fraction of 30% for 0.5 h each time to extract three times. The extracting solution was combined, filtered, and concentrated to obtain an extract with a relative density of 1.18-1.20. An appropriate amount of dextrin were as added (adding the extract and dextrin at a mass ratio of 1:1), and dried in an oven at 120° C., and then pulverized to obtain the extract.

### Embodiment 3: Preparation of HJT 60% Ethanol Extract and its Experimental Samples

HJT was purchased from Wuzhou City, Guangxi Province. The dry whole plant of HJT was taken, washed to remove the weeds and soil of the grass, and sliced into pieces of about 1-2 cm. About 500 g of the dry whole plan was extracted with 12 times the amount of ethanol aqueous solution with a volume fraction of 60% for 0.5 h each time to extract three times. The extracting solution was combined, filtered, and concentrated to obtain an extract with a relative density of 1.18-1.20. An appropriate amount of dextrin was added (adding the extract and dextrin at a mass ratio of 1:1), and dried in an oven at 120° C., and then pulverized to obtain the extract.

### Embodiment 4: Preparation of HJT 95% Ethanol Extract and its Experimental Samples

HJT was purchased from Wuzhou City, Guangxi Province. The dry whole plant of HJT was taken, washed to remove the weeds and soil of the grass, and sliced into pieces of about 1-2 cm. About 500 g of the dry whole plant was extracted with 12 times the amount of ethanol aqueous solution with a volume fraction of 95% for 0.5 h each time to extract three times. The extracting solution was combined, filtered, and concentrated to obtain an extract with a relative density of 1.18-1.20. An appropriate amount of dextrin was added (adding the extract and dextrin at a mass ratio of 1:1), and dried in an oven at 120° C., and then pulverized to obtain the extract.

### Embodiment 5: Preparation of HJT Extract with Different Polarities and its Experimental Samples

HJT was purchased from Wuzhou City, Guangxi Province. The dry whole plant of HJT was taken, washed to remove the weeds and soil of the grass, and sliced into pieces of about 1-2 cm. About 500 g of the dry whole plant extracted with 12 times the amount of boiled water for 0.5 h each time to extract three times. The extracting solution was combined, filtered, and then separated on a pretreated macroporous resin column (immersed in 95% ethanol for 24 hours). Then, water, the resin column was eluted sequentially with clear water, ethanol aqueous solution with a volume fraction of 20% and ethanol aqueous solution with a volume fraction of 60% at a flow rate of 3 BV/h. Each eluent was eluted for 3 column volumes, and the eluents were collected and concentrated to obtain 126.97 g of water eluent, 176.39 g of ethanol eluent with a volume fraction of 20%, and 132.05 g of ethanol eluent with a volume fraction of 60%.

### Embodiment 6: Observation of the Curative Effect of HJT Extract in Preventing and Treating the Blue-Ear Disease

1. Sample Processing:

(1) Astragalus polysaccharide: purchased from Henan Huakang Muda Agricultural Technology Co., Ltd.

(2) Qingwen Baidu powder: purchased from Shanghai Yingcai Animal Pharmaceutical Co., Ltd.

(3) HJT aqueous extract: Same as Embodiment 1, 1 g of powder equivalent to 3.0 g of raw medicinal materials was prepared.

(4) HJT 30% ethanol extract: Same as Embodiment 2, 1 g of powder equivalent to 3.0 g of raw medicinal materials was prepared.

(5) HJT 60% ethanol extract: Same as Embodiment 3, 1 g of powder equivalent to 3.0 g of raw medicinal materials was prepared.

(6) HJT 95% ethanol extract: Same as Embodiment 4, 1 g of powder equivalent to 3.0 g of raw medicinal materials was prepared.

2. Experimental Method:

Except for the normal control group, the other groups were given mixture to observe the effect on blue ear disease.

3. Experimental Process:

175 piglets (both male and female) infected with blue ear disease were randomly divided into 7 groups: normal control group, astragalus polysaccharide treatment group, Qingwen Baidu powder treatment group and 4 HJT extract treatment groups of the invention patent. At the same time, 175 adult pigs (both male and female) infected with blue ear disease were randomly assigned to the same seven groups. The group of astragalus polysaccharide was dosed with 30 g powder per pig per day. The group of Qingwen Baidu powder was dosed with 10 g powder per pig per day. Each pig in each group in the present invention was dosed with 20 g per day for 10 consecutive days. Data from piglets and adult pigs given the same drug were combined for statistics. On the 11th day after treatment, the number of cured heads, effective heads and ineffective heads in each group were counted, and the effective rate of each drug was calculated (the number of cured heads and effective heads were used to calculate the effective rate).

4. Criterion:

Cured: The pig has normal body temperature, no diarrhea, normal breathing, no edema, normal spirit and appetite.

Significant effect: The pig has normal body temperature, no diarrhea, mild respiratory symptoms, normal spirit and appetite.

Ineffective: The pigs died or failed to meet improvement standards.

5. Experimental Results:

As shown in Table 1, the overall effective rate of the HJT extract in the present invention is above 86%, and compared with the blank control group, the effective rate has improved by 80%, indicating that the HJT extract has a significant therapeutic effect on Blue-ear disease.

The SPSS 13.0 software was used to carry out statistical analysis on the test data, the group of HJT extract was better than the control drugs Astragalus polysaccharide and Qingwen Baidu powder, wherein the group of HJT aqueous extract had the highest overall effective rate. Therefore, the research on the anti-PRRS virus effect of HJT extract will be further carried out around the HJT aqueous extract.

TABLE 1
Observation of Curative Effect on Blue-ear Disease

| | Treatment Group for Piglets | | | Treatment Group for Adult Pigs | | | |
|---|---|---|---|---|---|---|---|
| | Quantity | Quantity of | Quantity | Quantity | Quantity of | Quantity | overall |
| | of being | significant | of being | of being | significant | of being | effective |
| Group | cured | effect | ineffective | cured | effect | ineffective | rate (%) |
| normal control | 0 | 0 | 25 | 0 | 3 | 22 | 6% |
| group | | | | | | | |
| Astragalus | 5 | 14 | 6 | 7 | 14 | 4 | 80% |
| polysaccharide | | | | | | | |
| Qingwen Baidu | 6 | 12 | 7 | 7 | 13 | 5 | 76% |
| powder | | | | | | | |
| HJT aqueous | 13 | 10 | 2 | 17 | 7 | 1 | 94% |
| extract | | | | | | | |
| HJT 30% | 9 | 13 | 3 | 13 | 10 | 2 | 90% |
| ethanol extract | | | | | | | |
| HJT 60% | 8 | 14 | 3 | 11 | 11 | 3 | 88% |
| ethanol extract | | | | | | | |
| HJT 95% | 9 | 13 | 3 | 11 | 12 | 3 | 88% |
| ethanol extract | | | | | | | |

### Embodiment 7: Observation of Cytotoxicity Study of HJT Extracts with Different Polarities

1. Materials

AlamarBlue (purchased from Invitrogen), used as an indicator of living cells metabolism, is converted to measurable fluorescent metabolites in response to enzymatic mitochondrial reduction reaction. Therefore, the cell activity can be monitored by measuring its absorbance.

2. Test Method

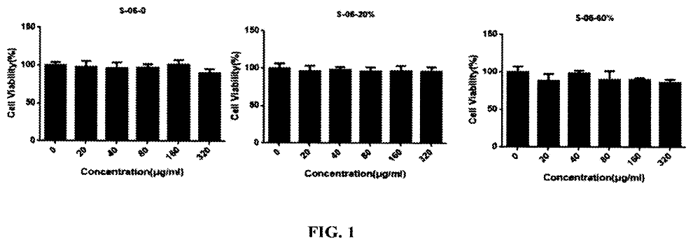

Marc-145 cells were cultured in a 96-well plate in DMEM medium containing 10% fetal bovine serum until the cell confluency was about 60-70%, the culture liquid was discarded, and 100 μL (dissolved in PBS) solution of the HJT extracts with different polarities (final concentrations being 20, 40, 80, 160, and 320 μg/ml) as prepared in Embodiment 5 of the present invention were respectively added to react for 24 h. The PBS control group was set, and then 10% (v/v) rate of AlamarBlue was added for 3 h. A multi-function microplate reader was used to read the absorbance values at 540 nm and 590 nm respectively, and then the cytotoxicity of different concentrations of HJT was obtained (as shown in FIG. 1).

The cell activity of the PBS control group was taken as 100%, the absorbance value of the cells treated with HJT extracts of different polarities and different concentrations were compared with the absorbance value of the PBS control group.

3. Results

It could be seen from FIG. 1 that when the concentration of HJT extract was 320 μg/ml and below, it had no toxicity upon Marc-145 cells, and the cell activity was 100%. Therefore, the concentration of HJT extract in subsequent experiments shall not be higher than 320 μg/ml.

### Embodiment 8: Study on Antiviral Test of HJT Extracts with Different Polarities Against PRRSV at Different Multiplicity of Infection (MOIs)

Marc-145 cells were cultured in a 12-well plate in DMEM culture medium containing 10% fetal bovine serum until the cell confluency was about 80%, the culture liquid was discarded, and the cells were washed for 3 times with PBS. 1 mL of DMEM culture medium containing 2% fetal bovine serum and 50 μL of the HJT extracts with different polarities as prepared in Example 5 of the present invention were added, and then inoculated with PRRSV-EGFP strains at different multiplicity of infection (MOIs). After the cells were cultured at 37° C. for 24 hours, the antiviral effect of HJT extracts with different polarities were observed under a fluorescence microscope.

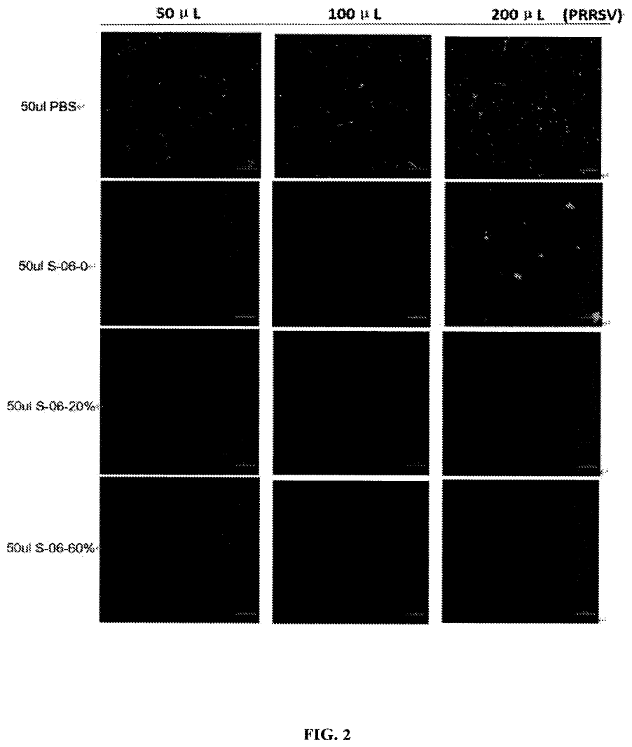

As shown in FIG. 2, the result was that HJT extracts with different polarities had strong inhibitory effect on PRRSV with different MOIs, but with the increase of MOIs, the inhibitory effect of water eluent of HJT aqueous extract on PRRSV decreased.

### Embodiment 9: Study on Antiviral Test of HJT Extracts with Different Polarities at Different Concentrations Against PRRSV

1. Marc-145 cells were cultured in a 12-well plate in DMEM culture medium containing 10% fetal bovine serum until the cell confluency was about 80%, the culture liquid was discarded, and the cells were washed for 3 times with PBS. 1 mL of DMEM culture medium containing 2% fetal bovine serum was added, then inoculated with PRRSV-EGFP at a multiplicity of infection MOI=0.1. After that, 10 μL, 50 μL and 100 μL of HJT aqueous extract, 20% ethanol eluent and 60% ethanol eluent as prepared in Embodiment 5 were respectively added so as to make different concentrations (1, 5, 10 μg/ml) as prepared in Embodiment 5, and PBS was added as a control. After the cells were cultured at 37° C. for 24 hours, the antiviral effect of HJT extracts with different polarities were observed under a fluorescence microscope.

2. Marc-145 cells were cultured in a 12-well plate in DMEM culture medium containing 10% fetal bovine serum until the cell confluency was about 80%, the culture liquid was discarded, and the cells were washed for 3 times with PBS. 1 mL of DMEM culture medium containing 2% fetal bovine serum was added, then inoculated with PRRSV at a multiplicity of infection MOI=0.1. After that, 25 μL, 50 μL and 100 μL of HJT aqueous extract, 20% ethanol eluent and 60% ethanol eluent as prepared in Embodiment 5 were respectively added so as to make different concentrations (1, 5, 10 μg/ml) as prepared in Embodiment 5, and PBS was added as a control. Mock referred to a blank control group, that has not been inoculated with virus. After the cells were cultured at 37° C. for 24 h, the cells were washed for 3 times with PBS, and the antiviral effect of HJT extracts with different polarities were detected by qRT-PCR for collecting cells.

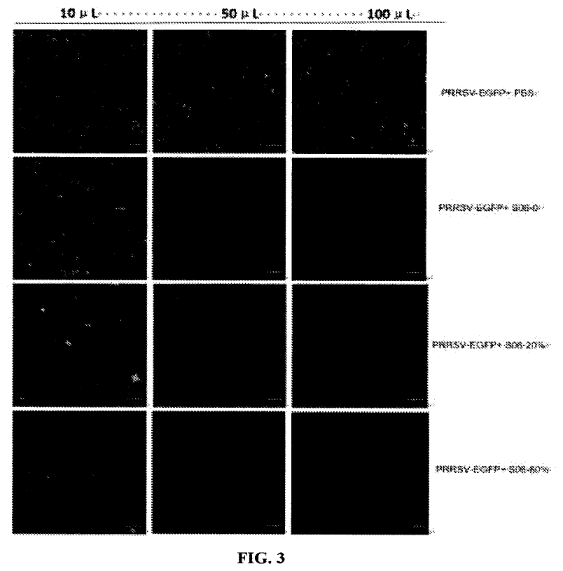

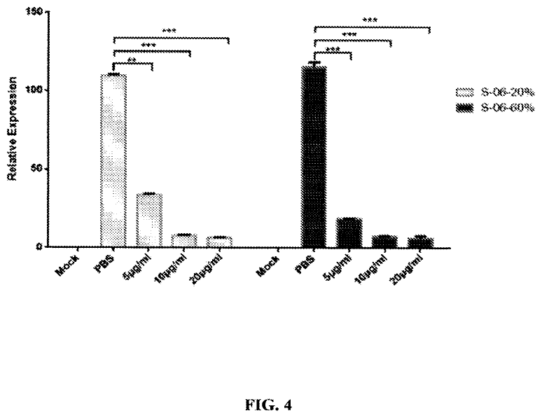

As shown in FIG. 3 and FIG. 4, the result was that HJT extracts with different polarities at different concentrations had strong inhibitory effect on PRRSV (MOI=0.1), and the inhibitory effect was dose-dependent. At low concentrations, ethanol eluent with a volume fraction of 60% of HJT aqueous extract had stronger inhibitory effect on PRRSV than ethanol eluent with a volume fraction of 20%.

What is claimed is:

1. A method of an application of a Hypericum japonicum thumb aqueous extract, a Hypericum japonicum thumb ethanol extract, or a Hypericum japonicum thumb ethanol aqueous solution extract in a preparation of a drug against a Porcine Reproductive and Respiratory Syndrome Virus.

2. The method of claim 1, wherein the application is in a preparation of drugs against a Blue-ear disease.

3. The method of claim 1, wherein the Hypericum japonicum thumb aqueous extract, the Hypericum japonicum thumb ethanol extract, or the Hypericum japonicum thumb ethanol aqueous solution extract is prepared by extracting a Hypericum japonicum thumb herb with water, ethanol, or an ethanol aqueous solution, and the water, the ethanol, or the ethanol aqueous solution is recovered to produce the Hypericum japonicum thumb aqueous extract, the Hypericum japonicum thumb ethanol extract, or the Hypericum japonicum thumb ethanol aqueous solution extract.

4. The method of claim 3, wherein a volume fraction of the ethanol aqueous solution is 30%, 60%, or 95%.

5. The method of claim 1, wherein the Hypericum japonicum thumb aqueous extract or the Hypericum japonicum thumb ethanol aqueous solution extract is obtained by adding a decocted solution of the Hypericum japonicum thumb herb into a macroporous adsorption resin column, sequentially eluting the macroporous adsorption resin column with a water and an ethanol aqueous solution, and recovering the water and the ethanol aqueous solution respectively to obtain the Hypericum japonicum thumb aqueous extract or the Hypericum japonicum thumb ethanol aqueous solution extract.

6. The method of claim 5, wherein the eluting the macroporous adsorption resin column with the water and the ethanol aqueous solution is conducted by using the water, an ethanol aqueous solution with a volume fraction of 20%, and an ethanol aqueous solution with a volume fraction of 60% in sequence.

7. A drug against a Porcine Reproductive and Respiratory Syndrome Virus, wherein a Hypericum japonicum thumb aqueous extract, a Hypericum japonicum thumb ethanol extract, or a Hypericum japonicum thumb ethanol aqueous solution extract is used as an active ingredient.

8. The drug against the Porcine Reproductive and Respiratory Syndrome Virus of claim 7, wherein the drug is against a Blue-ear disease.

9. The drug against the Porcine Reproductive and Respiratory Syndrome Virus of claim 7, wherein a dosage form of the drug is a powder, an oral liquid, or an injection liquid.

10. The drug against the Porcine Reproductive and Respiratory Syndrome Virus of claim 8, wherein a dosage form of the drug is a powder, an oral liquid, or an injection liquid.
